(19) United States
(12) Patent Application Publication
(10) Pub. No.: US 2003/0186431 A1
(43) Pub. Date: Oct. 2, 2003

(54) Rabbit hemorrhagic disease vaccine and antigens

(76) Inventors: Jose Rivera Torres, Madrid (ES); Francisco Rodriguez, Madrid (ES); Maria D. Fernandez, Madrid (ES); Juan Antonio Garcia Alvarez, Madrid (ES); Juan Plana-Duran, Olot (ES); Mercedes Mourino, Olot (ES)

Correspondence Address: WYETH, PATENT LAW GROUP, FIVE GIRALDA FARMS, MADISON, NJ, 07940, US

(21) Appl. No.: 10/363,503

(22) PCT Filed: Aug. 30, 2001
(86) PCT No.: PCT/EP01/10026

(30) Foreign Application Priority Data
Sep. 1, 2000 (ES) P200002161

(51) Int. Cl. A61K 39/12; C12N 7/01; C12N 15/82; C12N 15/86; C12N 7/00; A01H 1/00; C12N 15/87; C12N 15/00; C12N 15/09; C12N 15/63; C12N 15/70; C12N 15/74

(52) U.S. Cl. 435/320.1; 424/186.1; 435/235.1; 800/288; 424/199.1

(57) ABSTRACT

The VP60 structural antigen or a fragment therof of rabbit hemorrhagic disease virus (RHDV) can be obtained in plants using a viral vector based on the plum pox virus (PPV), which includes a promoter, a recombinant DNA sequence including a cDNA to the genome of the PPV virus, full-length, and a DNA sequence which codes for the RHDV VP60 protein or a fragment thereof inserted between the nucleotide sequences coding the Nib and CP proteins of the PPV virus, and a cloning vehicle. The antigen obtained is capable of inducing protection in rabbits against a lethal challenge of RHDV, and can be used as a recombinant subunit vaccine against rabbit hemorrhagic disease virus.

### SCOPE OF THE INVENTION

This invention pertains to the production of the structural antigen VP60, or a fragment thereof, from rabbit hemorrhagic disease virus in plants using a viral vector based on plum pox virus and the use of these antigens as a recombinant subunit vaccine against rabbit hemorrhagic disease virus.

### BACKGROUND OF THE INVENTION

Rabbit hemorrhagic disease (RHD) is a rapidly lethal infection of adult animals. Infected rabbits usually die of necrotizing hepatitis within 48 to 72 hours postinfection. The causal agent of the disease, rabbit hemorrhagic disease virus (RHDV), is a member of the family Calciviridae (Ohlinger et al., 1990. J Virol 64, 3331-3336; Parra & Prieto, 1990. J Virol 64, 4013-4015).

Existing commercial vaccines against RHD are produced from tissues such as spleen or liver from SPF rabbits experimentally infected with RHDV due to the fact that to date there is no permissible stable cell line allowing replication of said virus. In recent years, VP60, the major structural protein of RHDV, has been produced in several heterologous systems (Bárcena et al., 2000. J Virol 74, 1114-1123; Bertagnoli et al., 1996. J Virol 70, 5061-5066; Boga et al., 1994. J Gen Virol 75, 2409-2413; Boga et al., 1997. J Gen Virol 78, 2315-2318; Fischer et al., 1997. Vaccine 15, 90-96; Laurent et al., 1994. J Virol 68, 6794-6798; Marin et al., 1995. Virus Research 39, 119-128; Nagesha et al., 1995, Arch Virol 140, 1095-1108; Plana-Duran et al., 1996. Arch Virol 141, 1423-36; Sibilia et al., 1995. J Virol 69, 5812-5815), including production in transgenic plants (Castañón et al., 1999. J Virol 73, 4452-4455). In all cases, the recombinant VP60 protein obtained has been shown to be capable of protecting rabbits against a lethal challenge with RHDV.

At present, plant experiments are aimed at developing new systems for producing biological vaccines of interest. Plants offer a number of advantages over other expression systems. In particular, they present a safe, easy, and economical means of obtaining proteins of interest without the need for expensive scaling-up equipment, and they can replace exhausted traditional crops. Production by plants of proteins with pharmaceutical value and of proteins that can be used as subunit vaccines is of particular interest.

There are 2 main strategies for producing molecules of interest in plants: (i) genetic transformation of the plants' nuclear genome to create transgenic plants, and (ii) manipulation of plant virus genomes (Amtzen, 1997. Nat Biotechnol 15, 221-222). The latter strategy offers the advantage of permitting plants to produce relatively large quantities of the desired product at a certain point in their development cycle, and the viral stocks can be maintained for long periods of time without being passed through plants.

A number of systems have been developed for expression of heterologous proteins from a DNA genome virus as well as from a RNA genome virus (Scholthof et al., 1996. Ann Rev Phytopathol 34, 299-323). In the development of this invention, we used a PPV-NK expression vector. This vector was constructed from the plum pox virus, PPV (Fernández-Fernández, Doctoral Dissertation “Plum pox virus as an expression vector in plants.” Department of Molecular Biology. College of Sciences. University of Madrid (April 1999)) and is the object of the Spanish Patent Application No. P9900698 (Fernández-Fernández et al., 1999), which describes the construction of a recombinant DNA which comprises a cDNA to the genome of the PPV virus, a foreign sequence inserted between the coding sequences of the NIb and CP proteins and 2 recognition targets for 2 restriction enzymes (NaeI and KpnI), and it is useful for the construction of a heterologous protein expression vector in plants.

The PPV virus is a member of the potyvirus group which infects plants. The helical PPV virions are made up of a molecule of messenger RNA surrounded by more than 2,000 copies of the single capsid protein (CP) (Riechmann et al., 1992. J Gen Virol 73, 1-16). The RNA molecule has a viral protein called VPg covalently bonded to its 5′-end and a polyadenylate tail on its 3′-end. The expression strategy of the polyviruses' genome consists of translation into a large polyprotein which is processed by viral proteases to give rise to the final viral protein products.

### SUMMARY OF THE INVENTION

This invention pertains, generally, to the problem of providing a recombinant subunit vaccine against rabbit hemorrhagic disease virus, particularly with the production of an antigen that can be used as a vaccine against said disease.

The solution provided by this invention is based on the production of the RHDV VP60 structural antigen or a fragment thereof in plants or in plant cells using a viral vector based on PPV virus, specifically a PPV-NK expression vector. The production of these antigens is illustrated in Example 1, while the capacity of the antigens obtained to induce protection in rabbits against a lethal challenge with RHDV is illustrated in Example 2.

The use of an expression vector based on the PPV virus, which follows an expression strategy based on the production of a single polyprotein subsequently processed by viral proteases to give rise to the final viral protein products, presents the advantage that all the proteins, including the heterologous protein, are synthesized in the same quantity, and the differences in the levels of accumulation depend exclusively on their stability in infected plants or plant cells.

Therefore, one subject of this invention is a process for the production of the RHDV VP60 structural antigen or a fragment thereof in plants or plant cells that can be infected by the PPV virus; this includes the stage of constructing an expression vector of the RHDV VP60 structural antigen or a fragment thereof based on the PPV virus which includes the nucleotide sequence coding for RHDV VP60 antigen or a fragment thereof inserted between the nucleotide sequences coding for the NIb and CP proteins of the PPV virus. This expression vector of the RHDV VP60 antigen or a fragment thereof, based on the PPV virus, is also another subject of this invention.

An additional subject of this invention is a plant or a plant cell which can be infected by the PPV virus, which includes said expression vector of the RHDV VP60 antigen or a fragment thereof, based on the PPV virus.

Another additional subject of this invention is a recombinant subunit vaccine against rabbit hemorrhagic disease which includes said RHDV VP60 antigen or a fragment thereof obtained in plants using said expression vector based on the PPV virus. The procedure for production of said recombinant subunit vaccine is an additional subject of this invention.

### BRIEF DESCRIPTION OF THE FIGURES

FIG. 1 shows a western blot test of extracts from Nicotiana clevelandii plants inoculated with PPV-NK-VP60 (lanes 1-15) or with wild PPV (lane 16) using polyclonal serum against RHDV VP60 protein. The plants in lanes 3, 5, 8, and 14 were asymptomatic (data not shown). The bands in lanes 3 and 8 are probably due to overflow from the adjacent wells. The molecular weight markers used (BioRad) are indicated at the side of the panel.

### DETAILED DESCRIPTION OF THE INVENTION

The invention provides an expression vector of the RHDV VP60 antigen or a fragment thereof based on the PPV virus, referred to as the expression vector of the invention, which comprises:

a promoter

a recombinant DNA sequence including a cDNA to the PPV genome, full length, and a DNA sequence coding for the RHDV VP60 protein or a fragment thereof inserted between the nucleotide sequences coding for the proteins Nib and CP of PPV, and

a cloning vehicle.

As used in this description, the term “RHDV VP60 antigen (or protein), or a fragment thereof” means a protein which includes all or part of VP60 from RHDV, regardless of the RHDV isolate, capable of binding specifically with a T-cell receptor or antibody. In a particular embodiment, said protein is capable of provoking an immune response in the animal to which it is administered. The term “full length” means a complete genome sequence and included in this definition are the variants of RHDV VP60 which are differentiated from native proteins by addition, substitution, or deletion of amino acids, provided that they are capable of provoking this immune response.

Promoter

The promoter, or transcription promoter sequence, is a DNA sequence located at the 5′-terminal end and immediately anterior to nucleotide 1 of the cDNA of the PPV virus, to which the RNA polymerase binds to initiate RNA transcription. Said promoter may be:

an appropriate promoter for the in vitro transcription of the cDNA by means of the corresponding RNA polymerase, such as a bacteriophage promoter, e.g., from the T7 bacteriophage; or,

a promoter of a functional gene in plants, appropriate for the in vivo transcription of viral RNA, e.g., the 35S promoter of cauliflower mosaic virus (CaMV).

Recombinant DNA Sequence

The recombinant DNA sequence present in the expression vector of the invention, which includes a cDNA to the PPV virus genome, full length, and a DNA sequence coding for the RHDV VP60 protein or a fragment thereof inserted between the nucleotide sequences coding the NIb and CP proteins of the PPV virus, so that the product coded for by said coding sequence of RHDV VP60 protein or fragment thereof is expressed by forming part of the PPV polyprotein in the corresponding host between said NIb and CP proteins without producing changes in any of the viral proteins, so that there is as little interference as possible with any viral function. The native protease NIa is responsible for separating the RHDV VP60 protein or fragment thereof from the rest of the viral product.

The recombinant DNA sequence which includes a cDNA to the PPV virus genome, full length, and a DNA sequence coding the RHDV VP60 protein or fragment thereof inserted between the coding nucleotide sequences of the proteins NIb and CP of PPV, can be obtained by manipulation of a full-length cDNA clone of PPV virus, which is obtained by inverse transcription of the PPV genome by the process described in Spanish Patent Application No. P9800623.

In order to facilitate insertion of the DNA sequence coding for the RHDV VP60 protein or fragment thereof in the appropriate position on the cDNA of the PPV virus, a full-length clone of cDNA from the PPV virus can be manipulated by conventional genetic engineering techniques in order to introduce between the coding nucleotide sequences of the NIb and CP viral proteins of the PPV virus, a nucleotide sequence made up of the following elements, operatively linked, in the following order (according to the 5′→3′ direction of the coding chain):

a) a foreign sequence of nucleotides selected from:

i) a foreign sequence of 18 nucleotides making up the last 18 nucleotides of the coding sequence of the NIb protein and which code the first 6 amino acids of a recognition heptapeptide of the NIa protease from PPV, and

ii) a foreign sequence of 21 nucleotides coding a recognition heptapeptide of the NIa protease of PPV;

b) a nucleotide sequence which comprises a single recognition site of a first restriction enzyme;

c) a second spacer made up of at least 3 nucleotides of any kind in order to facilitate anchoring and to increase the efficacy of the restriction enzymes; and

d) a nucleotide sequence which comprises a single recognition site of a second restriction enzyme.

According to this construction, between the cistrons of the NIb and CP proteins of the PPV virus, there are 2 nucleotide sequences which code for a recognition heptapeptide of the NIa protease of PPV, and between both sequences there is a target for a first restriction enzyme and a target for a second restriction enzyme, both a single site. The foreign heptapeptide may be, for example, the heptapeptide NVVVHGA, a recognition heptapeptide of the NIa protease of PPV between the NIb and CP proteins of PPV.

In a particular embodiment, said foreign recognition sequence of the NIa protease of PPV is formed by the combination of (i) the foreign sequence of 18 nucleotides which constitute the last 18 nucleotides of the sequence coding the NIb protein and which code for the first 6 amino acids of a recognition heptapeptide of the NIa protease of PPV, and (ii) the first 3 nucleotides of the recognition sequence of the first restriction enzyme which code for the last amino acid of said recognition heptapeptide of the NIa protease of PPV, while the other nucleotide sequence which codes for said recognition sequence of the NIa protease of PPV is formed by (i) the 18 nucleotides immediately adjacent to the nucleotide sequence coding the CP protein, which corresponded initially to the last 18 nucleotides coding for the last 6 amino acids of the carboxyl end of the NIb protein of PPV, in combination with (ii) the first 3 nucleotides of the CP protein coding sequence which code for the last amino acid of said recognition heptapeptide of NIa protease of PPV.

In another particular embodiment, one of said recognition sequences of the NIa protease of PPV is formed by said foreign sequence of 21 nucleotides which codes for a recognition heptapeptide of the NIa protease of PPV, while the other nucleotide sequence which codes for said recognition sequence of the NIa protease of PPV is formed by (i) the 18 nucleotides immediately adjacent to the coding nucleotide sequence of protein CP, which correspond initially to the last 18 nucleotides which code for the last 6 amino acids of the carboxyl end of the NIb protein of PPV, in combination with (ii) the first 3 nucleotides of the sequence coding the CP protein, which code for the last amino acid of said recognition heptapeptide of NIa protease of PPV.

In a particular and preferred embodiment of this invention, the nucleotide sequences which code for both recognition sequences of the NIa protease of PPV are different, and they differ in one or more nucleotides, with the purpose of preventing or reducing possible recombination phenomena between homologous sequences that could lead to loss of the nucleotide sequence coding for the heterologous protein. In a particular embodiment, said nucleotide sequences code for the recognition heptapeptide of the NIa protease of PPV between NIb and CP, and they differ in at least one nucleotide in each triplet.

The introduction of the two restriction enzyme recognition targets or sequences into the cDNA clone of the PPV virus, full length, permits cloning of the DNA sequence which codes for RHDV VP60 protein or fragment thereof. In general, said first and second restriction enzymes can be any restriction enzyme. In a particular embodiment, the target for the first restriction enzyme is a target for the enzyme NaeI adjacent to the sequence coding for the carboxyl end of the NIb protein of PPV. In another particular embodiment, the target for the second restriction enzyme is a different target than the target for said first restriction enzyme, for example, a target for the enzyme KpnI.

Manipulation of a full-length clone of cDNA of the PPV virus in order to obtain the clones identified as pUC18-NK and pICPPV-NK has been described previously (Fernández-Fernández, 1999 as given above).

Cloning Vehicle

The cloning vehicle is a DNA molecule which possesses a replication origin and is therefore capable of replicating in a suitable cell.

Obtaining the Expression Vector of the Invention

The expression vector of this invention can be obtained by a process which includes digesting a clone of cDNA, full length, from PPV virus (manipulated in order to introduce the previously-mentioned nucleotide sequence a)-d) between the nucleotide sequences coding the NIb and CP viral proteins of the PPV virus), with the restriction enzymes whose recognition sequences are encountered in said manipulated cDNA clone, ligating the sequence coding RHDV VP60 or a fragment thereof, and inserting said recombinant DNA sequence of PPV which contains the VP60 sequence or fragment thereof into a cloning vehicle, optionally under the control of a suitable promoter.

Example 1 describes the construction of an expression vector of the RHDV VP60 antigen based on the PPV virus.

The invention also provides a process for obtaining the RHDV VP60 protein or a fragment thereof in plants or plant cells that can be infected by PPV, in a further procedure of the invention, which comprises:

1) inoculating said plants or plant cells susceptible to infection by PPV with the expression vector of the invention;

2) expressing said RHDV VP60 protein or a fragment thereof in said plants or plant cells contained in the viral polyprotein; and, optionally,

3) isolating said RHDV VP60 protein or a fragment thereof separated from the viral polyprotein by the proteolytic processing of said polyprotein.

A non-limiting illustrative list of some plant species that can be infected by PPV is given below:

1. Stone fruits: Prunus amygdalus, P. amygdalo-persica, P. armeniaca, P. avium, P. cerasifera, P. cerasus, P. cistena, P. domestica, P. mahaleb, P. mume, P. persica, P. spinosa, P. tomentosa, P. triloba.

2. Herbaceous plants

a) Chenopodiaceae: Cheniopodium capitatum, C. foetidum, C foilosum, C. quinoa

b) Compositae: Emilia sagittata, Senecio viscostis, S. vulgaris, Zinnia elegans

c) Labiatae: Lamium amplexicaule, L. purpureum, Galeopsis segetum

d) Papilionaceae: Vicia sativa ssp. angustifolia, Pisum sativum

e) Passifloraceae: Passiflora foetida

f) Ranunculaceae: Ranunculus arvensis, R. sardous

g) Scrophulariaceae: Linaria cymbalaria

h) Solanaceae: Nicandra physaloïdes, Nicotiana affinis, N. auriculata, N. clevelandii, N. debneyi, N. exigua, N. gigantea, N. glutinosa, N. knightiana, N. langsdorfii, N. longiflora, N. maritima, N. megalosiphon, N. noctiflora, N. nudicaulis, N. paniculata, N. plumbaginifolia, N. pomopsiflora, N. quadrivalvis N. repanda, N. rustica, N. solanifolia, N. sylvestris, N. tabacum, Solanum cornatum, S. luteum, S. miniatum, S. nigrum, S. nudiflorum, S. rostratum, S. sinaicum, S. sodomaeum, S. villosum, Petunia hyvrida, Physalis floridana,

Inoculation of the plants or plant cells susceptible to infection by PPV with the expression vector of this invention can be realized by any conventional method, e.g., mechanically or using the methodology of the Helios “gene-gun” (BioRad), or with any other technique suited for the transfer of genetic material to a plant.

In another particular embodiment of the procedure of the invention, it is possible to use an expression vector of the invention in which the recombinant DNA sequence containing the sequence coding RHDV VP60 or a fragment thereof is under a suitable promoter for in vitro transcription of DNA, in which case the RNA transcripts are obtained using a suitable RNA polymerase, e.g., the RNA polymerase from the T7 bacteriophage.

The RHDV VP60 protein or a fragment thereof, if desired, once it is separated from the viral polyprotein obtained by the proteolytic processing of this polyprotein, can be isolated from the medium and, if desired, purified by conventional methods.

If a whole plant is used for the production of the RHDV VP60 protein or a fragment thereof, the expression vector of the invention should be, preferably, defective in the capacity to be transmitted by aphids in order to prevent transmission of said recombinant vector to other plants. This can be achieved by inactivating the viral components necessary for insect transmission. For example, in the amino-terminal region of the CP protein of potyviruses, there is the triad of amino acids DAG (aspartic acid, alanine, and glycine) which is essential to transmission of the virus by aphids. Natural mutants of the PPV virus, called NAT (non-aphid transmissible) have been described (Maiss et al., 1992. J Gen Virol 73, 709-713; López-Moya et al., 1995. J Gen Virol 76, 2293-2297). These mutants have a deletion of 15 amino acids at the amino-terminal end of the CP protein, including deletion of the glycine from the DAG triad, which is essential for aphid transmission. A construction is available of a complete cDNA from PPV, called pGPPV-NAT, which harbors a mutation equivalent to the one which appears in the NAT mutants (Fernández-Fernández et al., 1998. FEBS Lett. 427, 229-235) from which it is possible to synthesize transcripts that are infectious in plants with the same characteristics as infection by wild transcripts. In a particular embodiment, for large-scale exploitation of the expression vector of the invention, said vector could harbor this mutation, which would make it biologically safe.

Therefore, in a particular embodiment, the sequence of cDNA from PPV present in the expression vector of the invention contains a NAT-type deletion to prevent aphid transmission of the recombinant viral vector to other plants.

An illustrative nonlimiting example describes the production of the protein VP60 from RHDV in Nicotiana clevelandii plants infected with the expression vector pICPPV-VP60 (Example 1).

The invention also provides plant cells that can be infected by PPV, which contain an expression vector of the invention capable of expressing the RHDV VP60 protein or a fragment thereof.

The invention also provides plants that can be infected by PPV, inoculated with an expression vector of the invention capable of expressing and accumulating RHDV VP60 protein or a fragment thereof.

The structural antigen RHDV VP60, or a fragment thereof obtained by the process of this invention is capable of inducing protection in rabbits against a lethal burden with RHDV, as shown in Example 2.

Therefore, the invention provides a recombinant subunit vaccine against the rabbit hemorrhagic disease virus, henceforth vaccine of the invention, which includes a therapeutically effective quantity of RHDV VP60 structural antigen or a fragment thereof obtained by the process of the invention, optionally combined with an adjuvant and/or a diluent.

In a particular embodiment, the vaccine of the invention contains an adjuvant such as an oily adjuvant made up of a mixture of Marcol-52, Simulsol-5100, and Montanide-888.

The vaccine of the invention can be prepared in any appropriate form of administration for administration to host animals, e.g., rabbits. In a particular embodiment, the administration of the vaccine of the invention to said animals is realized by the parenteral route, for example, by subcutaneous injection. In another particular embodiment, the administration of the vaccine of the invention to said animals is realized by the oral route.

The vaccine of the invention can be obtained by a method which includes (a) obtaining the RHDV VP60 structural antigen or a fragment thereof by the process of the invention, (b) separating an extract which includes an antigen phase containing said RHDV VP60 structural antigen or a fragment thereof, and optionally (c) mixing said antigen phase with an adjuvant and/or with a diluent.

Obtaining the RHDV VP60 structural antigen or a fragment thereof by the process of the invention, includes inoculation of plants or plant cells susceptible to infection by PPV or with the expression vector of the invention, and the expression of said RHDV VP60 protein or a fragment thereof in said plants or plant cells contained in the viral polyprotein from which the RHDV VP60 antigen or a fragment thereof is separated by the proteolytic process of the polyprotein.

The separation of the antigenic phase which contains the RHDV VP60 structural antigen or a fragment thereof can be realized by conventional methods, which include homogenization, from said plant or cell parts in a liquid medium, e.g., an aqueous liquid medium, and separation of the cellular debris in order to obtain said antigenic phase which contains the RHDV VP60 structural antigen or a fragment thereof. In a particular embodiment, we homogenize plant leaves or cells expressing the RHDV VP60 antigen or a fragment thereof in the presence of a phosphate-buffered saline solution CBS), and the separation of the cellular debris is realized by centrifugation. We obtain an antigen phase which contains RHDV VP60 antigen or a fragment thereof which is subsequently mixed with an oily adjuvant.

Example 2 describes the preparation of plants infected with an expression vector of the invention as an antigen source, the formulation of vaccines and the immunization of rabbits with said antigen and protection of the rabbits against a lethal burden with RHDV.

### EXAMPLE 1

Construction of the Expression Vector pICPPV-NK-VP60

1.1. Construction of the Expression Vector pICPPV-NK-VP60

The sequence which codes for the RHDV VP60 structural protein was amplified by PCR (from a plasmid containing its pUC-VP60 sequence (Rivera Torres, 1998. Alternatives to classic vaccination against myxomatosis and hemorrhagic disease in the European rabbit (Oryctotagus cuniculus). In Molecular Biology, Madrid: University of Madrid) using a polymerase with a low error rate (Expand^{R} High-fidelity, Boehringer Mannheim). The oligodeoxynucleotides used in the amplification were those identified as SEQ ID No. 1 and SEQ ID No. 2. After amplification, the PCR product, purified from an agarose gel, was treated with T4 DNA polymerase in order to leave the ends of the fragment blunt, and the product was then treated with the restriction enzyme KpnI, since the oligonucleotide used in the amplification and identified as SEQ ID No. 2 creates a site for said target in the end of the amplified fragment. We used the intermediate plasmid pUC18-NK (Fernández-Fernández, 1999 as given above) in order to facilitate the chimera construction process. The plasmid was digested with the restriction enzymes NaeI and KpnI and after this treatment, they were ligated with the PCR product containing the VP60 gene treated as described above. The resulting plasmid is called pUC18-NK-VP60. The EcoRV-KpnI fragment of the plasmid pUC18-NK-VP60 was introduced into the pICPPV-NK clone (Fernández-Fernández, 1999, as given above) by triple ligation using the XhoI enzyme as the third enzyme in order to give rise to the complete chimeric pICPPV-NK-VP60 clone, which was deposited in the CECT [see section on DEPOSIT OF MICROORGANISMS]. The complete cDNA genome of PPV was located, cloned in pICPPV-NK under the control of the promoter 35S of the cauliflower mosaic virus (López-Moya & Garcia, 2000. Virus Research 68, 99-107), which allows for the production of in vivo viral transcripts in plants directly inoculated with DNA.

1.2. Inoculation of Plants

The full-length cDNA present in the plasmids constructed from the pICPPV clone (wild or mutant) was diluted to a concentration of 250 ng/μl, and the plants were inoculated with 10 μl of these dilutions for a total of 3 leaves per plant, previously sprinkled with carborundum (silicon carbide). Alternatively, the Helios “gene-gun” (BioRad) methodology could be used for inoculation of the plant with quantities which can amount to as low as 10 ng per plant (López-Moya & Garcia, 2000, as above).

The plants inoculated with the pICPPV-NK-VP60 clones were infected. The course of the infection and its symptoms were similar to those of plants inoculated with the wild pICPPV clone.

1.3. Western Blot Analysis

Samples from infected plants homogenized with 5 mM sodium phosphate buffer, pH 7.5, were separated by polyacrylamide gel electrophoresis with sodium dodecyl sulfate (SDS-PAGE). The samples were transferred to a nitrocellulose membrane and incubated with a polyclonal antibody against RHDV VP60 protein according to the protocol described in (Garcia et al., 1992. Virology 188, 697-703). The second antibody used was goat anti-rabbit IgGs, conjugated with peroxidase (Jackson Immunoresearch Laboratories). The peroxidase reaction was developed with the ECL™ kit (Amersham Pharmacia Biotech).

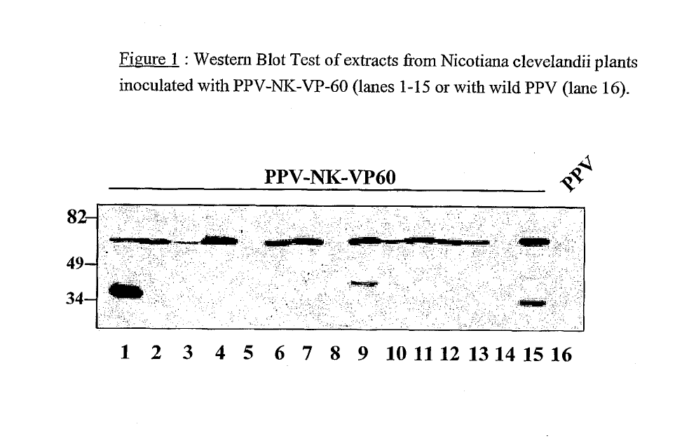

The protein VP60 was readily detected by western blot tests 15 days postinoculation (d.p.i.); the highest levels of accumulation were reached 21 d.p.i. (FIG. 1). Fifteen d.p.i., no other band could be detected apart from that of the complete protein by western blot tests with an anti-VP60 antibody (data not shown), however, 21 d.p.i. bands were apparent with electrophoretic mobility corresponding to truncated proteins of approximately 38 kDa (plant 1), 44 kDa (plant 9), and 46 and 33 kDa (plant 15), in anti-VP60 western blot tests (FIG. 1). Although we cannot rule out the possibility that some of these bands correspond to degraded protein, it seems likely that most of said bands are the result of a certain instability of the chimeric genome. This assumption was corroborated by the detection of partial deletions of the heterologous gene in a viral cDNA fragment amplified by immunocapture-polymerase chain reaction (IC-PCR) from infected tissues (data not shown). In any case, the complete RHDV VP60 protein was the predominant form in most of the plants, demonstrating the system's capacity to efficiently express proteins with a practical interest of certain size.

### EXAMPLE 2

Recombinant Subunit Vaccine Against RHD

2.1. Formulation of Vaccines

Leaves from plants infected with either wild PPV or with PPV-NK-VP60 [chimera (virus) generated from the expression vector pICPPV-NK-VP60] were homogenized in PBS (in a ratio of 1:1 or 1:2 w/v) and the cellular debris were eliminated by centrifugation. The vaccines were formulated by mixing the antigenic phase (leaf extracts) with an oily adjuvant in a ratio of 53:47 (v/w) until a double water/oil/water emulsion was formed. The oily phase consisted of a mixture of Marcol-52, Simusol-5100, and Montanide-888. We prepared 4 different plant-based vaccines. We also used an inactivated commercial vaccine which was available for the prevention of rabbit hemorrhagic fever as an experimental control, together with an oily adjuvant (CYLAP HIVD, Fort Dodge Veterinaria). These vaccines were administered parenterally by subcutaneous injection.

2.2. Immunization of Rabbits With Extracts From Plants Infected With PPV-NK-VP60, and Protection Against a Challenge With RHDV

We studied the immunogenicity of the RHDV VP60 protein expressed in plants with the vector pICPPV-NK-VP60 in the natural host for RHDV, rabbits. Twenty-seven days after the first administration of antigen, the serum samples were analyzed for the presence of specific antibodies by means of the HI test (see Table 1) (the test consists of the determination of the level of antibodies against RHDV in the serum of rabbits by means of a human erythrocyte hemagglutination inhibition test). Nine of the 10 rabbits in group I (vaccinated with extracts from plants infected with PPV-NK-VP60 expressing the complete protein VP60) exhibited specific VP60 antibodies with variable titers which ranged from 1/20 to 1/320. Similar antibody titers were observed in the animals of group III (vaccinated with a plant extract which accumulated, in addition to the complete VP60 protein, a deleted form of approximately 44 kDa). Three of the 4 rabbits of group II (vaccinated with a plant extract which mostly accumulated a deleted form of 38 kDa, in addition to the complete VP60 protein) gave a lower serological response (titers between 1/20-1/40). The antibody response was somewhat higher and more homogeneous in the animals vaccinated with the commercial vaccine CYLAP HVD (Fort Dodge, Veterinaria) (group V) (with animals showing titers between 1/320-1/640). As expected, the animals vaccinated with the extract coming from a plant infected with wild PPV (group IV) and the unvaccinated control group (group VI) showed no specific antibodies against RHDV. Thirty-three days after the initial immunization (D33), all the animals were given a booster. On day 67 (D67), the antibody titers were analyzed by means of the HI test and the animals were inoculated with a lethal challenge of RHDV. While the control groups (IV and VI) remained negative, the serological response increased in all the other vaccination groups. Again, the highest titers were detected in the groups vaccinated with CYLAP HVD and with whole protein VP60 (groups V and I), followed by the groups vaccinated with the plants that also accumulated deleted forms of the VP60 protein (group III works better than group II).

The challenge was adequate, as the mortality rate from RHDV in the group vaccinated with the plant infected by the wild PPV virus and the unvaccinated group was 100%, and RHDV was detected in their livers (Table 1). During the period of experimental infection with the virulent RHDV, while all except one of the animals of control groups IV and VI died within the first 3 days after the challenge (the other died 5 days afterwards), no clinical symptoms of the disease were observed in the animals vaccinated with CYLAP HVD (group V) or with the plants infected with PPV-NK-VP60 (groups I, II, and III). Two weeks after the challenge, the surviving animals were bled and slaughtered. In general, the antibody titers in the surviving animals, as determined by the HI test, were higher 2 weeks after the challenge (Table 1). No RHDV virus was detected in the livers of the surviving animals. Therefore, we can conclude that the animals vaccinated with the plant extracts which expressed the VP60 protein, exclusively in whole form, or in deleted forms as well, developed a specific humoral immune response and were protected against a lethal challenge with RHDV.

Conclusions

1. RHDV VP60 structural protein has been expressed in a PPV-NK expression vector which expresses the sequence coding for said protein of interest between the cistrons which code for protein NIb and CP from PPV.

2. The chimera PPV-NK-PV60 has the same infectivity characteristics as the wild virus.

3. Immunization of rabbits (natural hosts of RHDV) with extracts coming from plants infected with PPV-NK-VP60 chimera induces in them a specific and efficient antibody response against RHDV. The animals are protected against a lethal challenge with RHDV.

TABLE 1
Results of antibody titers against RHDV and the lethal challenge

| | | | | Results of the challenge | | |
|---|---|---|---|---|---|---|
| | | | | | | HA titer |
| | Animal | HI titers^{c} | | | HI Titers | in liver |
| Group^{a} | No. | D27^{b} | D67 | Death | DY + 15 | samples^{d} |
| Group I | 818 | 1/320 | 1/2560 | S | 1/10240 | <2 |
| PPV-NK-VP60 | | | | | | |
| (whole VP60, | | | | | | |
| plants | | | | | | |
| 2 + 4 + 6 + 7 + 11 + 13) | | | | | | |
| | 821 | 1/160 | 1/2560 | S | 1/5120 | <2 |
| | 822 | 1/80 | 1/640 | S | 1/2560 | <2 |
| | 823 | 1/80 | 1/1280 | S | 1/5 120 | <2 |
| | 826 | 1/20 | 1/640 | S | 1/5 120 | <2 |
| | 827 | 1/20 | 1/320 | S | 1/160 | <2 |
| | 831 | <1/20 | 1/1280 | S | 1/2560 | <2 |
| | 834 | 1/40 | 1/160 | S | 1/2560 | <2 |
| | 837 | 1/80 | 1/640 | S | 1/5120 | <2 |
| | 897 | 1/160 | 1/5120 | S | 1/2560 | <2 |
| Group II | 845 | 1/40 | 1/320 | S | 1/5120 | <2 |
| PPV-NK-VP6oΔ1 | | | | | | |
| (whole and | | | | | | |
| deleted VP60 of | | | | | | |
| ˜38 kDa, plant 1) | | | | | | |
| | 846 | <1/20 | 1/160 | S | 1/1280 | <2 |
| | 847 | 1/40 | 1/320 | S | 1/640 | <2 |
| | 848 | 1/20 | 1/320 | S | 1/1280 | <2 |
| Group III | 839 | 1/20 | 1/320 | S | 1/640 | <2 |
| PPV-NK-VP6oΔ9 | | | | | | |
| (whole and | | | | | | |
| deleted VP60 of | | | | | | |
| ˜44 kDa, plant 9 | | | | | | |
| | 840 | 1/320 | 1/1280 | S | 1/5120 | <2 |
| | 842 | 1/20 | 1/640 | S | 1/10240 | <2 |
| | 844 | 1/40 | 1/640 | S | 1/2560 | <2 |
| Group IV | 900 | <1/20 | <1/20 | DY + | | ≧4096 |
| PPV-wt (without | | | | 3 | | |
| insertion, plant | | | | | | |
| 16) | | | | | | |
| | 809 | <1/20 | <1/20 | DY + | | ≧4096 |
| | | | | 3 | | |
| | 813 | <1/20 | <1/20 | DY + | | ≧4096 |
| | | | | 3 | | |
| | 816 | <1/20 | <1/20 | DY + | | ≧4096 |
| | | | | 3 | | |
| Group V | 851 | 1/320 | 1/5120 | S | 1/10240 | <2 |
| CYLAP HVD | | | | | | |
| (Fort Dodge | | | | | | |
| Vaccine) | | | | | | |
| | 853 | 1/320 | 1/2560 | S | 1/5120 | <2 |
| | 854 | 1/640 | 1/2560 | S | 1/10240 | <2 |
| | 855 | 1/320 | 1/640 | S | 1/10240 | <2 |
| Group VI | 804 | <1/20 | <1/20 | DY + | ≧4096 | |
| Control | | | | 3 | | |
| (unvaccinated | | | | | | |
| | 824 | <1/20 | 1/20 | DY + | | ≧4096 |
| | | | | 3 | | |
| | 825 | <1/20 | 1/20 | DY + | | ≧4096 |
| | | | | 3 | | |
| | 863 | <1/20 | <1/20 | DY + | | ≧4096 |
| | | | | 5 | | |
| | 864 | <1/20 | <1/20 | DY + | | ≧4096 |
| | | | | 3 | | |
| | 866 | <1/20 | <1/20 | DY + | | ≧4096 |
| | | | | 3 | | |

^{a} Plants are numbered according to FIG. 1. ^{b} The rabbits were vaccinated on day 0 (D0), were bled on day 27 (D27). They had a booster on day 33 (D33), were bled again on day 67 (D67), and a challenge on the same day (DY). The surviving animals (S) were bled and slaughtered 2 weeks after the challenge (DY+15). ^{c}HI Test: the level of antibodies against RHDV in the rabbits' serum was determined by a human erythrocyte hemaglutination inhibition test. The rabbits were seronegative for RHDV on D0 (HI titers<1/20). ^{d}HA Test: the presence of RHDV was determined by human erythrocyte hemagglutination. Titers<2 are considered negative.

Deposit of Microorganisms

Cells from Esherichia coli DH5α contained in the plasmid pICPPV-NK-VP60 were deposited in the Spanish Collection of Type Cultures (CECT), Buljasot, Valencia (Spain) on Aug. 8, 2000, corresponding to access number CECT 5348.

List of Sequences:

(1) GENERAL INFORMATION

(1) APPLICANT

(1) NAME: BOARD OF SCIENTIFIC RESEARCH

(2) STREET: 113 Serrano

(3) CITY: Madrid

(4) PROVINCE: Madrid

(5) COUNTRY: ES

(6) POSTAL CODE: 28006

(7) TELEPHONE: 91 585 50 00

(8) FAX: 91 441 30 77

(1) APPLICANT:

(1) NAME: FORT DODGE VETERINARIA, S.A.

(2) STREET: Carretera de Camprodón s/n “La Riba”

(3) CITY: Vall de Bianya

(4) PROVINCE: Girona

(5) COUNTRY: ES

(6) POSTAL CODE: 17813

(7) TELEPHONE: 972 29 00 01

(8) FAX: 97229 0102

(2) TITLE OF THE INVENTION:

RABBIT HEMORRHAGIC DISEASE VACCINE AND ANTIGENS.

(3) NUM3ER OF SEQUENCES: 2

(4) INFORMATION FROM SEQ. ID. NO. 1:

(1) SEQUENCE CHARACTERISTICS:

(1) LENGTH: 18bases

(2) TYPE: Nucleic acid

(3) NUMBER OF CHAINS: single

(4) TOPOLOGY: linear

(2) TYPE OF MOLECULE: Nucleic acid

(xi) DESCRIPTION OF THE SEQUENCE: SEQ. ID. NO. 1:

ATGGAGGGCAAAGCCCGC

(2) INFORMATION FROM SEQ. ID. NO. 2:

(2) SEQUENCE CHARACTERISTICS:

(1) LENGTH: 23 bases

(2) TYPE: Nucleic acid

(3): NUMBER OF CHAINS: single

(4) TOPOLOGY: linear

(2) TYPE OF MOLECULE: Nucleic acid

(xi) DESCRIPTION OF THE SEQUENCE: SEQ. ID. NO. 2:

CAGGTACCGACATAAGAAAAGCC

1. An expression vector of the VP60 antigen of rabbit hemorrhagic disease virus (RHDV) or a fragment thereof, based on the plum pox virus (PPV), which comprises:
a promoter
a recombinant DNA sequence which includes a cDNA to the genome of the PPV virus, full length, and a DNA sequence which codes for RHDV VP60 protein or a fragment thereof inserted between the nucleotide sequences coding the NIb and CP proteins of the PPV virus, and
a cloning vehicle.

2. Vector according to claim 1, in which said promoter is chosen from the group made up of a suitable promoter for in vitro transcription of cDNA by an RNA polymerase and a promoter of a functional gene in plants suitable for the in vivo transcription of the viral RNA.

3. Vector according to claim 2, in which said promoter is chosen from the group made up of the promoter of the T7 bacteriophage and the 35S promoter of cauliflower mosaic virus (CaMV).

4. Vector according to claim 1, which comprises a NAT mutation.

5. A process for obtaining VP60 protein of rabbit hemorrhagic disease virus (RHDV) or a fragment thereof in plants or plant cells that can be infected by the plum pox virus (PPV), which comprises:
1) inoculating said plants or plant cells susceptible to infection by PPV with an expression vector of the RHDV VP60 protein or a fragment thereof, based on the PPV virus, according to any of claims 1-4;
2) expressing said RHDV VP60 protein or a fragment thereof in said plants or plant cells, contained in the viral polyprotein; and, optionally,
3) isolating said RHDV VP60 protein or a fragment thereof separated from the viral polyprotein by means of the proteolytic processing of said polyprotein.

6. A plant cell susceptible to infection by the plum pox virus (PPV), which contains an expression vector of the RHDV VP60 protein or a fragment thereof based on the PPV virus, according to any of claims 1-4.

7. A plant susceptible to infection by the plum pox virus (PPV), which contains an expression vector of the RHDV VP60 protein or a fragment thereof based on the PPV virus, according to any of claims 1-4, capable of expressing and accumulating RHDV VP60 protein or a fragment thereof.

8. A recombinant subunit vaccine against rabbit hemorrhagic disease virus which includes a therapeutically effective quantity of RHDV VP60 structural antigen or a figment thereof obtained by a process according to claim 5, optionally together with an adjuvant and/or diluent.

9. Vaccine according to claim 8, which includes an adjuvant.

10. Vaccine according to claim 9, in which said adjuvant is an oily adjuvant made up of a mixture of Marcol-52, Simusol-5100, and Montanide-888.

11. Vaccine according to claim 8, prepared for administration to host animals of RHDV by the parenteral route.

12. Vaccine according to claim 11, prepared for administration to host animals of RHDV by the subcutaneous route.

13. Vaccine according to claim 8, prepared for administration to host animals of RHDV by the oral route.

14. A method for obtaining a recombinant subunit vaccine according to claim 8, which comprises (a) obtaining the RHDV VP60 structural antigen or a fragment thereof by a procedure according to claim 5, (b) separating an extract which includes an antigen phase containing said RHDV VP60 structural antigen or a fragment thereof, and optionally (c) mixing said antigen phase with an adjuvant and/or a diluent.

15. Method according to claim 14, in which the separation of said extract including said antigen phase which contains said RHDV VP60 structural antigen or a fragment thereof includes homogenization of the parts of the plants or cells which express the RHDV VP60 structural antigen or a fragment thereof in a liquid medium, and separation of the cellular debris.

16. Method according to claim 15, in which homogenization tales place in an aqueous liquid medium.

17. Method according to claim 15, in which the separation of said cellular debris is realized by centrifugation.
